(19) United States
(12) Patent Application Publication
(10) Pub. No.: US 2026/0315090 A1
(43) Pub. Date: Oct. 15, 2026

(54) DEVICE FOR APPLYING SOUND WAVES

(71) Applicant: Richard Wolf GmbH, Knittlingen (DE)

(72) Inventors: Anton GIETL, Bretten (DE); Dominic VALET, Bruchsal (DE); Stefan SÄLZLER, Östringen (DE); Michael BURKHARDT, Mühlacker (DE); André ANTONI, Ölbronn (DE)

(21) Appl. No.: 19/116,538

(22) PCT Filed: Sep. 29, 2023
(86) PCT No.: PCT/DE2023/200206

§ 371 Date: Mar. 28, 2025

(30) Foreign Application Priority Data
Sep. 30, 2022 (DE) 10 2022 210 443.9

(51) Int. Cl. A61B 17/225; A61B 17/00

(52) U.S. Cl. CPC A61B 17/2251; A61B 2017/00367; A61B 2017/2253

(57) ABSTRACT

A device is for the application of sound waves onto an animal and human body. The device includes a housing in which a fluid reservoir which is filled with a coupling fluid is formed. A sound wave source for generating sound waves which can be coupled into the body via a coupling membrane is arranged in the fluid reservoir. The fluid reservoir includes a sound wave generation region in which the sound wave source is arranged, and a bubble retention region which is provided for holding back gas bubbles. The bubble retention region is partly spatially separated from the sound wave generation region by way of a separating wall but there is a fluid communication between the regions. The sound wave source is arranged between the bubble retention region and the coupling membrane and is aligned such that sound waves are delivered in a direction of the coupling membrane.

### CROSS REFERENCE TO RELATED APPLICATIONS

This application is a United States National Phase Application of International Application PCT/DE2023/200206, filed Sep. 29, 2023, and claims the benefit of priority under 35 U.S.C. § 119 of German Application 10 2022 210 443.9, filed Sep. 30, 2022, the entire contents of which are incorporated herein by reference.

### TECHNICAL FIELD

The present invention relates to a device for the application of sound waves onto an animal or human body. The device comprises a housing in which a fluid reservoir for a coupling fluid is formed and a sound wave source for generating sound waves. The sound wave source is arranged in the fluid reservoir. Furthermore, the device comprises a coupling membrane which in sections forms a wall of the fluid reservoir and is provided for coupling the sound waves which are generated by the sound wave source, into a human or animal body.

### BACKGROUND

Devices for extracorporeal focussed medical sound waves treatment such as for example shock waves, burst waves or continuous waves have been known from the state of the art for some time now. The devices comprise a sound wave source which is arranged in a housing and which generates the focussed sound waves or acoustic waves, which can also be denoted as pressure waves or shock waves. As a rule, these sound waves are emitted onto a coupling medium such as for example a coupling membrane. A coupling surface of the device bears on a surface of the person or animal, into which the sound waves are to be coupled. The coupling surface can be part of the coupling membrane or be formed by additional gel pads which are used for the adaptation of the penetration depth of the sound waves into the body.

In order to change the penetration depth of the sound waves into the animal or human body, from the state of the art it is known to use coupling membranes or gel pads of a different thickness or width. On account of the differently thick coupling membranes, the distance between the human or animal body and the sound wave source can be changed and this has a direct effect on the penetration depth of the sound waves which are generated by the sound wave source.

In order to change the penetration depth of the sound waves, the device must be removed and refitted, which entails a certain amount of effort, to which the user often has an aversion. Moreover, the correct treatment position needs to be sought again after the refitting, by which means the treatment duration is extended. For this reason, the same configuration is often used for different indications and target areas, despite the fact that different penetration depths would be advantageous.

A device for the application of acoustic shock waves, concerning which a sound wave source is movably arranged in a housing of the device, is known from EP 1 520 536 A1. The housing comprises a coupling surface and the sound wave source can be moved relative to this. With the coupling membrane, the housing forms a closed-off volume in which a medium which is suitable for the propagation of the shock waves and which can also be denoted as a coupling fluid is introduced.

### SUMMARY

It is an object of the present invention to provide an improved device for the application of sound waves onto an animal or human body, concerning which the sound wave source is arranged in a coupling fluid in the housing of the device.

The object underlying the invention is solved by a device according to the invention. Preferred embodiments of the device are disclosed herein.

A device for the application of sound waves onto an animal and human body is provided for achieving the object, said device comprising a housing in which a fluid reservoir for a coupling fluid is formed. The device further comprises a sound wave source for generating sound waves. The sound wave source is arranged in the fluid reservoir. Furthermore, the device comprises a coupling membrane which forms a wall of the fluid reservoir and is provided for coupling the sound waves which are generated by the sound wave source, into a human or animal body. The sound wave source is arranged in a sound wave generation region of the fluid reservoir. The sound wave generation region is delimited by the coupling membrane at least in sections. The fluid reservoir furthermore comprises a bubble retention region for holding back gas bubbles, said bubble retention region being in fluid communication with the sound wave generation region and being spatially separated from this partly by way of a separating wall. The sound wave source is arranged between the bubble retention region and the coupling membrane and is aligned in a manner such that sound waves which are generated by the sound wave source are delivered away from the bubble retention region and onto the coupling membrane.

In other words, the present invention relates to a device for the extracorporeal sound wave treatment, for example by way of shock waves, burst waves or continuous waves, wherein the sound waves are preferably focussed and are to be brought into an animal or human body. The device firstly comprises a housing which can be shaped for example as a hand grip which the user takes in his hand in order to position the device at a suitable location on a body. The housing encompasses a sound wave source with which the sound waves can be generated, said sound waves to be introduced into the body or applied onto this. Such sound wave sources have been known to the man skilled in the art for some time from the state of the art.

Furthermore, the device comprises a coupling membrane which forms a part of the housing. The coupling membrane comprises for example a first and a second surface, wherein the first surface is aligned onto the sound wave source and the second surface forms a coupling surface which is provided for contact with the body, into which the sound waves are to be brought. The coupling membrane for example can be a gel pad.

The fluid reservoir comprises at least two regions, a sound wave generation region and a bubble retention region. Herein, the section of the fluid reservoir in which the sound wave source is arranged and which is delimited by the coupling membrane at least in sections is denoted as a sound wave generation region. This region is filled with a coupling fluid when the device is operating, by way of which fluid the sound waves which are generated by the sound wave source are coupled onto the coupling membrane and herewith onto the body. Gas bubbles arise in the coupling fluid on filling the fluid reservoir as well as on operation of the sound wave source. These gas bubbles alter the capability of the coupling fluid to couple the sound waves which are generated by the sound wave source, onto the coupling membrane. If for example gas bubbles are located in the coupling fluid between the sound wave source and the coupling membrane during the operation of the device, then the coupling of the sound wave source onto the coupling membrane worsens and less power is available for being coupled into the body.

The bubble retention region of the fluid reservoir is provided in order to prevent gas bubbles from being located and accumulating between the sound wave surface and the coupling membrane. The bubble retention region is in fluid communication with the sound wave generation region, which is to say that the coupling fluid and also the gas bubbles can flow or stream from the sound wave generation region into the bubble retention region and vice versa.

In order to hold back the gas bubbles, the bubble retention region is arranged in a section of the fluid reservoir which lies on the side of the sound wave source which is away from the coupling membrane. Herewith, the sound wave source is arranged between the bubble retention region and the coupling membrane and delivers its sound waves in the direction of the coupling membrane and not in the direction of the bubble retention region. Furthermore, a separating wall is provided, said separating wall separating the bubble retention region at least partly from the sound wave generation region. Preferred designs of the separating wall are the subject-matter of the subsequently preferred embodiments.

On operation of the device, it has been found that the device is usually held by the operator or user such that the coupling membrane is arranged below the sound wave source with regard to gravity. Since the bubble retention region in turn is arranged on the side of the sound wave source which faces away from the coupling membrane, this region is usually arranged above the sound wave source and the coupling membrane with regard to gravity. The gas bubbles which are located in the coupling fluid or are generated in this thus rise counter to gravity and collect in the bubble retention region. Herewith, one already prevents the gas bubbles from worsening the coupling characteristics of the coupling fluid between the sound wave source and the coupling membrane.

The additionally provided separating wall advantageously prevents the gas bubbles from being able to escape directly out of the bubble retention region into the sound wave generation region given an inclination of the device relative to gravity. Herewith, in an advantageous manner, one prevents the gas bubbles which have collected in the bubble retention region from escaping out of the bubble retention region again given an inclination of the device and compromising the coupling of the sound wave source from the coupling membrane through the coupling fluid.

The separating wall is preferably formed by an undercut which projects out of the wall of the fluid reservoir. The undercut can be shaped for example as an overhang or projection which is set onto the wall of the fluid reservoir or is indeed formed as part of the wall or of a section of the wall. In this case, the undercut would be designed as one piece or as one part with a part of the wall of the fluid reservoir. As is illustrated in the subsequent embodiments, the undercut can herein assume different shapes and fashions.

In a preferred embodiment, the separating wall extends annularly away from the wall of the fluid reservoir. If for example the separating wall is formed by a projection which projects out of the wall of the fluid reservoir, then in the preferred embodiment this projection extends annularly along the wall of the fluid reservoir. Herein, annularly does not imply that the reservoir has a circular or an elliptical cross section. In contrast, here any peripheral separating wall which extends away from the wall of the fluid reservoir is to be understood by the term annularly. Hence a projection which extends peripherally away from the wall of a fluid reservoir with a rectangular cross section would also be annular. The advantage of a separating wall which extends annularly away from the wall lies in the fact that the gas bubbles are reliably captured or collected behind the separating wall independently of the inclination of the device and cannot get between the sound wave source and the coupling membrane.

In a further preferred embodiment, the separating wall comprises a first section and a second section. The first section extends between the sound wave source and the bubble retention region. The second section connects onto the first section and extends away from the sound wave source and the coupling membrane.

In other words, the separating wall is constructed at least of a first section and a second section. The first section is herein shaped and arranged in the fluid reservoir such that it runs essentially between the sound wave source and the bubble retention region. The first section runs or extends between the sound wave source and the bubble retention region if for example the extension of the separating wall in the first section is larger in this direction than in a direction which points away from the sound wave source. For example, the first section of the separating wall could run parallel to an extension direction of the sound wave source or be inclined to this at an angle of less than 45°. Herein, the first section of the separating wall does not need to be designed in a plane manner, but can also have a curved course. If the separating wall is formed by a projection which extends away from the wall of the fluid reservoir, then for example the section of the separating wall which connects directly onto the outer wall of the fluid reservoir can be denoted as a first section. Herein, it is the wall of the fluid reservoir which surrounds this which is denoted as an outer wall here.

The second section of the separating wall connects onto the first, which is to say that the first section of the separating wall merges into the second section. This transition can be effected by an angled bend in the separating wall. However, it is just as conceivable to provide a continuous transition between the first and the second section. The second section of the separating wall is designed such that it extends away from the sound wave source and the coupling membrane. Herewith, the extension of the separating wall in the second section is larger in a direction which points away from the sound wave source and the coupling membrane than in a direction which runs parallel to the extension direction of the sound wave source and the coupling membrane. For this, the second section of the separating wall can be inclined for example at an angle of more than 45° to an extension direction of the sound wave source and the coupling membrane. Herein, the second section of the separating wall too does not need to be plane, but in particular can be curved. For example, the separating wall in a second section can firstly have an angle of 45° relative to an extension direction of the sound wave source and the coupling membrane and become further inclined with an increasing distance to the first section of the separating wall.

In particular, in the present embodiment, one does not necessarily assume that the separating wall necessarily needs to be designed as a projection on the outer wall of the fluid reservoir. In contrast, it is also conceivable for the separating wall to be arranged at a reservoir with an essentially circular cross section in the middle of the reservoir. The separating wall in this case could comprise for example a third section which firstly extends away from the outer wall of the fluid reservoir in the direction of the sound wave source and the coupling membrane. This section could be arranged for example centrally in the fluid reservoir on a surface of the outer wall which lies opposite the coupling membrane and possibly encompass a drive axle or a supply channel, via which the sound wave source can be displaced or however at least provided with energy. In this case, the first section would connect onto the third section and herewith for example extend away from the middle of the fluid reservoir to the outer walls.

In a further preferred embodiment, the wall of the fluid reservoir comprises a bleed opening, through which the fluid reservoir is bled, so that gas bubbles can escape out of the fluid reservoir through the bleed opening. The device furthermore comprises a closure means with which the bleed opening can be closed. The provision of a bleed opening is advantageous to a high degree since gas bubbles which arise for example on filling the fluid reservoir with a coupling fluid or on operation of the sound wave source can be discharged out of the fluid reservoir through this bleed opening. In particular, the device can also comprise several bleed openings. In an exemplary embodiment, the closure means is a valve which is permanently inserted into the bleed opening and is temporarily opened for bleeding the fluid reservoir and closed again, without the closure means having to be removed for this.

Preferably, the bleed opening runs out into the bubble retention region. In this manner, one can ensure that gas bubbles or air bubbles which have collected in the bubble retention region can be reliably discharged from the fluid reservoir. In particular, if only a single bleed opening is provided, it can be necessary for the device to have to be moved to and fro, so that all gas bubbles get to the bleed opening, for the complete or at least extensive bleeding of the fluid reservoir. Furthermore, for the complete or extensive bleeding of the fluid reservoir, in many cases it may be necessary to either refill coupling fluid or however to reduce the volume of the fluid reservoir. The latter can be achieved for example by way of pressure being exerted upon the coupling membrane from the outside, inasmuch as this is formed from a flexible material.

It is further preferable for the bleed opening and the closure means to be configured such that the closure means is brought into the bleed opening in a closure position for closing the bleed opening and is positioned only partly removed from the bleed opening in a bleed position for bleeding the fluid reservoir.

In other words, in the preferred embodiment one can envisage the closure means being brought into the bleed opening and being able to be removed from this again, at least partly. For example, the closure means can be a screw closure which is screwed into the bleed opening. For this, the bleed opening can be provided for example with a thread. The closure means then comprises the respective counter-thread.

In order to bleed the fluid reservoir, in the preferred embodiment the closure means is not completely removed again from the bleed opening, but only partly. For example, the closure means can be screwed out of the bleed opening again by a predefined number of revolutions or a predefined angle, inasmuch as the closure means is screwed into the bleed opening. A bleed channel opens on account of the closure means being at least partly removed out of the bleed opening again, said bleed channel for example being able to run through the closure means itself or however also past the closure means, as is yet illustrated in more detail in the scope of preferred embodiments.

Since in the preferred embodiment, the closure means is not completely removed from the bleed opening for bleeding the fluid reservoir, but in contrast remains in this, the closure means advantageously cannot get lost and furthermore a controlled bleeding is rendered possible.

In a preferred embodiment, the bleeding is effected through the closure means itself. For this, the closure means for example can comprise a channel which runs along its longitudinal axis and which comprises an opening at a side which faces away from the fluid reservoir. This channel which runs along the longitudinal axis can run out into one or more lateral openings at the opposite end. If the closure means is brought into or positioned in the bleed position, then the lateral opening or the lateral openings are in fluid communication with the fluid reservoir, so that the gas bubbles can escape out of the fluid reservoir through the channel in the closure means.

Alternatively, the closure means can also comprise a channel with a lateral outlet which in the bleed position is aligned for example with a recess or a bleed channel in the housing of the device. If simultaneously the second end of the bleed channel to the closure means, said second end being at the fluid reservoir side, is in fluid communication with the fluid reservoir, then the bleeding of the fluid reservoir can be effected through the bleed channel and the respective recess in the housing of the device.

Alternatively, it is also conceivable for the bleeding to be effected past the closure means when this is in the bleed position. For this, in the bleed position the closure means at least in sections can no longer be in sealing contact with the housing of the device, so that gas bubbles can escape out of the fluid reservoir through an intermediate space which arises on account of this. In order to permit a further escape of the gas bubbles, in particular a recess or a bore which forms a bleed channel can also be provided in the housing of the device and this channel can be brought into fluid communication with the fluid reservoir when the closure means is in the bleed position. Herein, the fluid communication according to this embodiment is not effected through the closure means, but past this.

In a further preferred embodiment, a sealing means is arranged in the bleed opening in a manner such that in the closure position the closure means is in sealing contact with the sealing means and that in the bleed position a bleeding through an intermediate space between the sealing means and the closure means is possible.

In other words, a sealing means, for example in the form of an O-ring, which is arranged in the bleed opening, is provided in the preferred embodiment. In the closure position, the closure means is held against this O-ring or the sealing means and by way of this seals the fluid reservoir. For example, the closure means can be screwed in against the sealing means and press this together. If the sealing means is brought from the closure position into the bleed position, then according to the preferred embodiment an intermediate space arises between the sealing means and the closure means, through which space gas bubbles can escape out of the fluid reservoir. In the preferred embodiment, it is not absolute as to which further path the gas bubbles take from the gas reservoir into the surroundings. For example, they can escape through the closure means and/or through an additional opening or recess in the housing of the device.

In a further preferred embodiment, the fluid reservoir can also be filled with coupling fluid through the bleed opening. For this, coupling fluid can be brought into the fluid reservoir through a feed conduit, for example given a partially or completely removed closure means. In particular, if no additional bleed opening is provided, then it can herein be necessary for coupling fluid to be filled into the fluid reservoir and gas bubbles to be removed from the fluid reservoir in an alternating manner through the bleed opening. For this, one can envisage pump movements being carried out with the membrane in the case of the use of a flexible membrane, thus firstly coupling fluid is filled into the fluid reservoir and the gas bubbles which arise with this are removed again through the bleed opening by way of pressure upon the coupling membrane. The air which is located in the fluid reservoir before the filling of this can also be removed from the fluid reservoir in this manner.

In an alternative exemplary embodiment, a filling opening is also provided apart from one or more bleed openings, through which filling opening the fluid reservoir can be filled with coupling fluid. This has the advantage that a bleeding through the additional bleed opening or the additional bleed openings can take place directly during the filling of the reservoir with coupling fluid.

In a preferred embodiment of the present device, the sound wave source is movably arranged in the fluid reservoir in a manner such that a distance between the sound wave source and the coupling membrane can be changed. An intermediate space between the sound wave source and the coupling membrane is filled with coupling fluid on operation of the device. In the preferred embodiment, one therefore envisages the sound wave source being movable. This has the advantage that the penetration depth of the sound waves into the human or animal body can be changed by way of changing the sound wave source position in the fluid reservoir and, entailed by this, the change of the distance between the coupling membrane or the coupling surface which is formed thereon, without having to deposit coupling membranes of different thicknesses for this. The region between the coupling membrane and the sound wave source is filled with coupling fluid on account of the calotte-shaped sound wave source, so that the sound waves can be coupled into the coupling membrane and herewith into the body to an improved extent.

In a further preferred embodiment, the device comprises a drive for changing the distance between the sound wave source and the coupling membrane. The drive can be a manual drive or an actuator which converts electrical signals into a mechanical movement of the sound wave source. For example, the actuator can be an electrical drive, a hydraulic drive or a pneumatic drive. Preferably, the drive is a spindle drive.

Thus one preferably envisages the device comprising an adjustment mechanism with which the distance between the sound wave source and the coupling membrane and herewith the penetration depth of the sound waves into the human or animal body can be adjusted. Basically, two different drive types are conceivable.

The drive preferably comprises an adjustment wheel which is configured such that the distance between the sound wave source and the coupling membrane can be changed by way of a manual rotation of the adjustment wheel. Further preferably, a planetary gear is provided, amid whose use a rotation of the adjustment wheel can be converted into a change of the distance between the sound wave source and the coupling membrane.

The drive can be effected either manually which is to say a user of the device actuates for example a setting wheel, with which the position of the sound wave source within the fluid reservoir can be changed. For example, an adjustment ring can be provided in the housing of the device. The adjustment ring and the associated drive can then be designed such that given a rotation of the adjustment ring, for example via a planetary gear and a spindle drive, the sound wave source is either moved in the direction of the coupling membrane or away from this. Such a manual drive permits a simple and robust adjustment of the position of the sound wave source and herewith of the penetration depth of the sound waves.

Preferably, the device comprises a spring-loaded engagement element which is biased in the direction of the adjustment wheel. The adjustment wheel furthermore comprises a plurality of indentations which are arranged at predefined distances. The engagement element and the indentations are arranged and configured such that depending on a position of the adjustment wheel, the engagement element engages into the indentations, so that an additional force is necessary in order to disengage the engagement element from engagement with the indentation by way of rotating the adjustment wheel. Each position of the adjustment wheel in which the engagement element engages with one of the indentations corresponds to a predefined distance between the sound wave source and the coupling membrane.

Preferably, the engagement element is biased radially away from the rotation axis of the adjustment wheel towards the adjustment wheel, or parallel to the rotation axis of the adjustment wheel towards the adjustment wheel.

Alternatively or supplementarily, it is possible to provide an electrical or electronic drive, concerning which for example an electric motor which is attached in the device can change the position of the sound wave source relative to the coupling membrane via a spindle drive. The control of the actuator can be effected for example via operating elements, such as for example switches, on the housing. However, it is also conceivable for the control of the electric motor to be effected externally via a signal or data lead. Thus for example it is possible for a computer to set the distance between the sound wave source and the coupling membrane on the basis of data or pictures of imaging or locating systems, such as for example ultrasound, X-ray, magnetic resonance imaging or computer tomography, in dependence on the position of the device on a human body.

In a further preferred embodiment, the coupling membrane forms a bulge of the fluid reservoir away from the sound wave source, wherein the sound wave source can be moved into the bulge. The coupling membrane therefore has a preferably convex shape which extends away from the sound wave source. Due to the convex shape, the coupling membrane forms an outward formation or bulge, in which the fluid reservoir has a greater dimension along a movement direction of the sound wave source. The sound wave source is advantageously configured such that it can be moved into the bulge which is formed by coupling membrane. The size of the housing can be reduced due to the thus gained, additional distance between the sound wave source and the coupling membrane, without herein reducing the maximally possible adjustment range of the penetration depth of the sound waves.

Herein, it is particularly preferable for the coupling membrane to have a hollow truncated cone shape which tapers in the direction away from the sound wave source and projects out of the housing of the device.

In the preferred embodiment, the coupling membrane including the volume which is encompassed by it herewith has the shape of a truncated cone, which is to say that including the encompassed volume it is trapezoidal in cross section. Herein, the coupling membrane is hollow, which is to say that on the surface which faces away from the coupling surface it comprises a recess or bulge which is filled with coupling fluid on operation of the device. Furthermore, the coupling membrane is designed such that the shape of the truncated cone shape tapers in the direction away from the sound wave source and herewith to the body, into which the sound waves which are generated by the sound wave source are to be introduced. The cross-sectional area of the coupling membrane including the volume which is enclosed by it therefore reduces in the direction from the sound wave source to the body.

Furthermore, in the preferred embodiment one envisages the coupling membrane projecting out of the housing of the device. Herewith, one advantageously ensures that it is only the coupling membrane which has a softer surface which lies on the human or animal body into which the sound waves are to be brought. In a particularly advantageous manner, the body does not therefore come into contact with the remaining housing of the device, said housing being formed for example of a stiff plastic. Moreover, by way of this one can also couple onto body locations which anatomically are particular difficultly accessible and an unevenness of the body surface is compensated to an improved extent.

The present invention is hereinafter explained in more detail with reference to the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a sectional view through an embodiment example of a device for the application of sound waves;

FIG. 2 is a schematic sectional view of a bleed opening with a closure means in a closure position;

FIG. 3 is a schematic sectional view of the bleed opening of FIG. 2 with a closure means in the bleed position;

FIG. 4 is a schematic sectional view of an alternative design of a bleed opening;

FIG. 5 is a schematic sectional view of a positioning aid for a rotary ring with which a penetration depth of sound waves can be set;

FIG. 6 is a schematic, perspective sectional view of an alternative positioning aid for a rotary ring, with which a penetration depth of sound waves can be set;

FIG. 7 is a perspective view of an embodiment example of a coupling membrane; and

FIG. 8 is a further schematic sectional view of an alternative design of a bleed opening.

### DESCRIPTION OF PREFERRED EMBODIMENTS

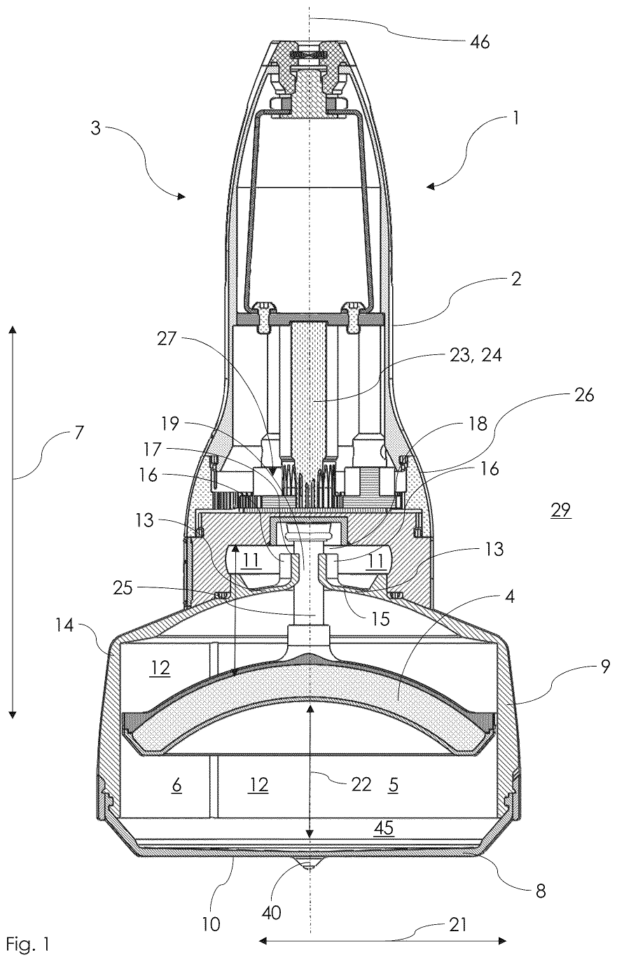

Referring to the drawings, FIG. 1 shows a sectioned view through an embodiment of a device 1 for the application of sound waves onto an animal or human body. The device comprises a housing 2. The device 1 is an ergonomically shaped hand-piece 3 with grip recesses. The hand-piece 3 is also denoted as a therapy source. A sound wave source 4 is arranged in the housing 2. Sound waves for the application onto an animal or human body are generated by the sound wave source 4. In the present embodiment example, the sound wave source 4 is a piezoelectric sound wave source 4 which generates sound waves for treating a human body.

The sound wave source 4 is arranged in a fluid reservoir 5 which is filled with a coupling fluid 6 for operation of the device 1. The sound wave source 4 can be moved along an adjustment direction 7 in the fluid reservoir 5, in order to adjust the penetration depth of the sound waves which are generated by the sound wave source 4, into the human body.

The device 1 further comprises a coupling membrane 8 which forms a part of the outer wall 9 of the housing 2 of the device 1. The coupling membrane 8 in particular comprises a coupling surface 10 which is laid or applied onto a surface of the body, in order to apply the sound waves.

The coupling membrane also forms a part of the wall of the fluid reservoir 5 and in particular delimits this in the direction of the body into which the sound waves which are generated by the sound wave source 4 are to be introduced.

On operation of the device 1, which is to say when sound waves are generated by way of the sound wave source 4 and introduced into a body, the fluid reservoir 5 is filled with a coupling fluid 6 which improves the coupling of the sound waves onto the coupling membrane 8 and thus also onto the body to be treated, said sound waves being generated by the sound wave source 4.

Degassed water, oils or alcohol can be used for example as coupling fluid, wherein in particular a conservation agent which combats the formation of germs can also be added to the de-gassed water. Other fluids are also suitable as a coupling fluid, inasmuch as these are good transmitters of sound waves, i.e. as little as possible gases and no air bubbles should be present. Moreover, no germs should form in the fluid, so that this fluid can be retained for as long as possible. Furthermore, the acoustic impedance of the coupling fluid must match that of the patient body, in order to reduce losses at the transition between the media. The fluid should also have a low acoustic damping.

Gas bubbles which can compromise the transmission of the sound waves from the sound wave source 4 to the coupling membrane 8 arise on filling the coupling fluid 6 into the fluid reservoir 5 as well as on operation of the sound wave source 4.

In order to improve the coupling of the sound wave source 4 onto the coupling membrane 8 and to keep the gas bubbles away from the region between the sound wave source 4 and the coupling membrane 8, the fluid reservoir 5 comprises a bubble retention region 11 which is at least partly spatially separated from a sound wave generation region 12 of the fluid reservoir 5. Here, the section of the fluid reservoir 5 in which the sound wave source 4 is arranged is denoted as a sound wave generation region 12.

The bubble retention region 11 is designed such that it receives and holds back gas bubbles which arise or are present in the coupling fluid 6, so that these bubbles do not get into the region between the sound wave source 4 and the coupling membrane 8 or only with great difficulty. For this, the bubble retention region 11 is firstly arranged in a section of the fluid reservoir 5 which lies at the side of the sound wave source 4 which faces away from the coupling membrane 8. In other words, the sound wave source 4 is arranged between the coupling membrane 8 and the bubble retention region 11. The background to this is the fact that on operation the device 1 is often held by an operator such that the coupling surface 8 is arranged below the sound wave source 4 with respect to gravity. Since the gas bubbles rise in the coupling fluid 6 counter to gravity, these move in the direction of the bubble retention region 11 on their own accord.

In order for the gas bubbles to be able to get from the sound wave generation region 12 into the bubble retention region 11, this retention region should not be completely spatially separated from the sound wave generation region 12, but must be in fluid communication with this. A separating wall 13 which is designed as an undercut and projects out of the wall 14 of the fluid reservoir 5 is provided in order to simultaneously hold back as many as possible gas bubbles in the bubble retention region 11, even if the device 1 is inclined at different angles to gravity.

The separating wall 13 is designed in an annular manner, thus projects out of the wall 14 in a circumferential manner, said wall in a cross section perpendicular to the adjustment device 7 of the sound wave source 4 otherwise having a circular cross section.

For capturing the gas bubbles, the separating wall 13 comprises a first section 15 and a second section 16, wherein the second section 16 connects onto the first section 15. The first section 15 runs essentially parallel to an extension direction 17 of the coupling membrane 8 and the sound wave source 4. This extension direction 17 furthermore runs perpendicularly to the adjustment direction 7 of the sound wave source 4. The first section 15 of the separating wall is consequently arranged between the bubble retention region 11 and the sound wave source 4.

The second section 16 of the separating wall 13 in contrast runs essentially parallel to the adjustment direction 7 and herewith away from the sound wave source 4 as well as the coupling membrane 8. In the embodiment of the device 1 which is shown in FIG. 1, only a narrow free space 18 is present between a free end 17 of the separating wall 13, on which end the second section 16 of the separating wall 13 ends, and the wall 14 of the fluid reservoir 5.

If on operation the device is held only slightly inclined to the direction of the gravity, then the gas bubbles in the coupling fluid 6 rise into the intermediate space 19 which is surrounded by the second section 16 of the separating wall 13 and flow into the bubble retention region 11 given a slight lateral tilting of the device 1 relative to gravity. The gas bubbles are reliably held back in this region and do not flow back into the sound wave generation region 12 even given a significant tilting of the device 1 relative to gravity. Herewith, the gas bubbles which have once gotten into the bubble retention region 11 are prevented from getting back into the sound wave generation region 12 and there being able to compromise the coupling of the sound wave source 4 onto the coupling membrane 8.

Furthermore, an adjustment mechanism is represented in FIG. 1, with which mechanism the position of the sound wave source 4 or the distance 22 of this to the coupling membrane 8 and herewith the penetration depth into the human body by sound waves which are generated by the sound wave source 4 can be adjusted. For this, a drive 23 in the form of a spindle drive 24 to which the sound wave source is connected via an axle 25 is provided in the embodiment example of the device 1 which is shown in FIG. 1. The distance 22 can be set by an operator of the device 1 by way of the operator rotating an adjustment wheel 26 which moves the spindle drive 24 via a planetary gear 27 which is not represented in more detail. Furthermore, supply leads for the sound wave source 4, for example for electrical energy, are led through the axle 25.

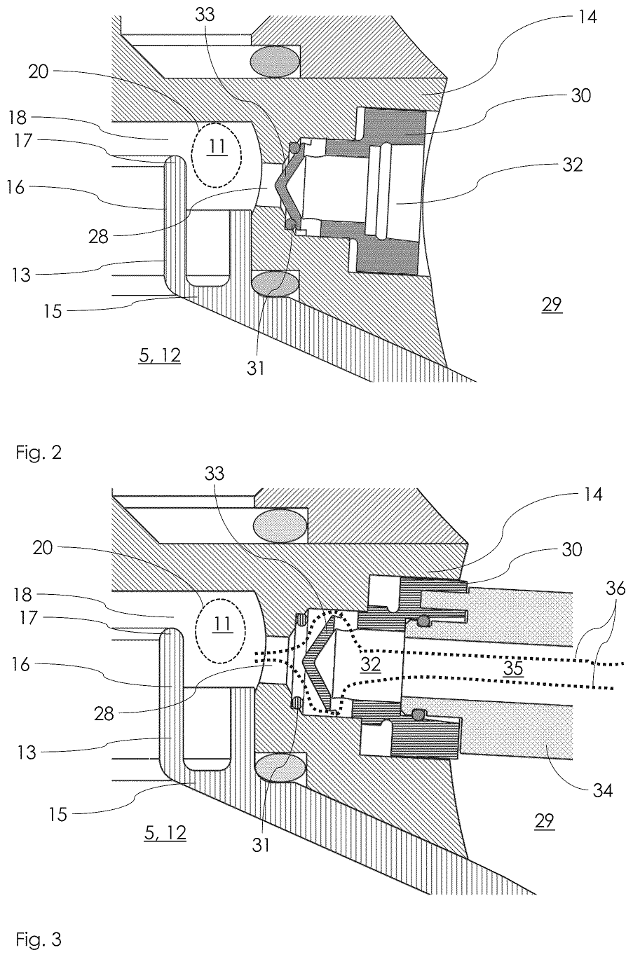

A bleed opening 28 is formed in the housing 2 of the device 1 in order to bleed the fluid reservoir 5, which is to say in order to remove gas bubbles 20 which are shown by way of example in FIG. 2, out of the bubble retention region 11. The bleed opening 28 is not represented in FIG. 1. However, two different embodiments of a bleed opening are shown in FIGS. 2 to 4 and these are described in more detail hereinafter.

Firstly, with reference to FIGS. 2 and 3, here a bleed opening 28 which connects the fluid reservoir 5 to the surroundings 29 of the device is shown. The bleed opening 28 is formed in the wall 14 in a manner such that it runs out into the bubble retention region 11. By way of example, an accumulation 20 of gas bubbles is represented in the bubble retention region 11. The device 1 further comprises a closure means 30 which can be screwed into the bleed opening 28 in order to close this closure means. If the closure means 30 is situated in a closure position as is shown in FIG. 2, then the closure means 30 is in contact with a sealing means 31 in the form of an O-ring. The contact between the closure means 30 and the sealing means 31 closes the fluid reservoir 5 in a sealing manner, so that neither gas bubbles nor coupling fluid can escape from this reservoir.

The closure means 30 is partly removed from the bleed opening 28, for example by way of it being screwed out of this closure means again by a certain distance, in order to bleed the fluid reservoir 5. A closure means 30 is shown in a respective bleed position in FIG. 3. In the embodiment which is shown in FIG. 3, the closure means 30 comprises a bleed channel 32 through which gas bubbles can escape from the fluid reservoir 5 when the tip 33 of the closure means 30 is no longer in contact with the sealing means 31.

A special tool 34 with which the closure means 30 can be screwed out of the bleed opening 28 and screwed into this again is additionally shown in FIG. 3. The special tool 34 is simultaneously used for filling and/or bleeding the fluid reservoir 5, wherein not only can gas bubbles escape through a channel 35 which is formed in the special tool 34, but fresh coupling fluid 6 can be filled into the fluid reservoir 5 through it. The flow paths which the gas bubbles take out of the fluid reservoir 5 or along which the coupling fluid 6 flows into the fluid reservoir 5 are provided with the reference numerals 36 in FIG. 3.

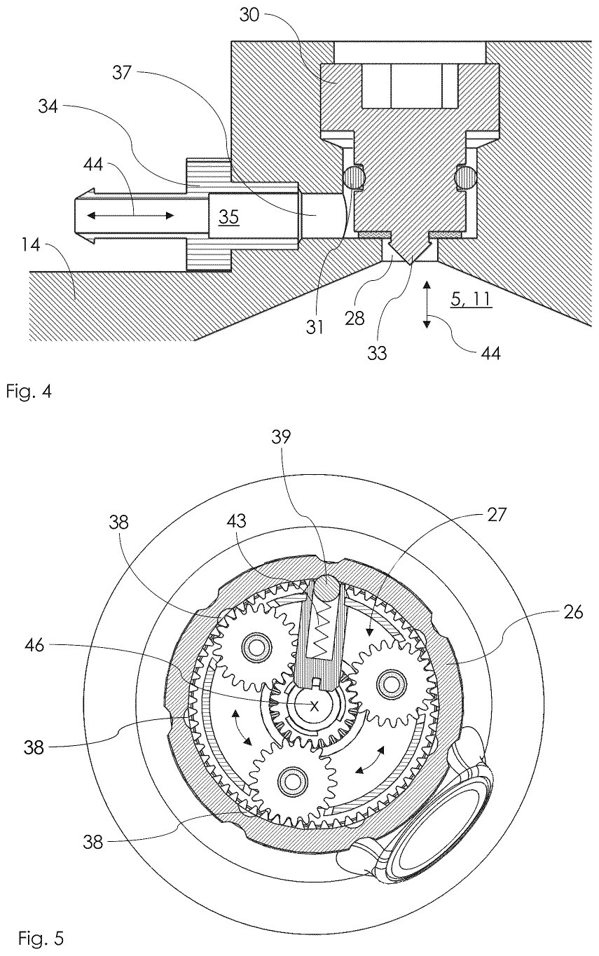

FIG. 4 shows an alternative design of a closure means 30 which is provided for closing a bleed device 28. In this case, the closure means 30 has no bleed channel. In contrast, for bleeding the fluid reservoir 5, by way of a tool 34 the closure means 30 is screwed so far out of the bleed opening 28 that the bleed opening 28 is in fluid communication with a further recess 37 in the wall 14 of the fluid reservoir. The bleeding is this case is effected through the part of the bleed opening 28 which faces the fluid reservoir 5 as well as of the recess 37 which is a direct part of the housing 2 of the device 1. The flow direction of the gas bubbles is represented in FIG. 4 by the arrows which are represented by the reference numerals 44.

Finally, a part of the adjustment device or of the drive 23 is represented in FIG. 5, with which part the adjustment wheel 26 can be locked in predefined positions and herewith the sound wave source 4 positioned at set distances to the coupling membrane 8. For this, several recesses, troughs or indentations 38 can be provided on the inner wall of the adjustment wheel 26, of which only three have been provided with a reference numeral for the purpose of a better overview.

A spring-loaded engagement element 39 which is radially biased in the direction of the adjustment wheel 26 by way of a spring 43 is provided within the device 1. In the embodiment example in FIG. 5, the engagement element 39 is spherical. If the adjustment wheel 26 is rotated, in order to bring the spindle drive 24 into rotation via the planetary gear 27, then the engagement element 39 engages into the recesses 38 at set distances. An additional force is then necessary in order to further rotate the adjustment wheel 26. Herewith, it is signalled to the user that he has reached a predefined depth. The depth can be indicated for example externally on the adjustment wheel 26 by way of lettering.

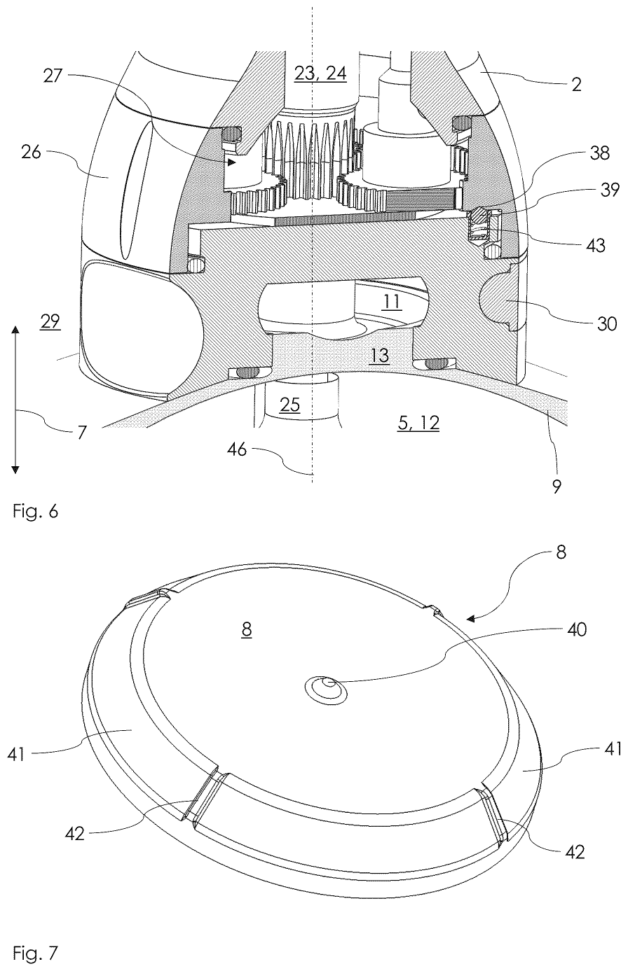

FIG. 6 shows an alternative design of an adjustment device which differs from the representation which is shown in FIG. 5 essentially in that the engagement means 39 is not biased in the plane of the planetary gear 27 but perpendicularly thereto, thus parallel to the direction of the axle 25 or parallel to the adjustment device 7. The spring 43 therefore biases the spherical engagement means onto the adjustment wheel 26 along the adjustment direction 7. The recesses are accordingly arranged differently in the adjustment wheel 26. The function of the adjustment device corresponds completely to that of the adjustment device which has already been described with reference to FIG. 5, so that the respective details are referred to, in order to avoid unnecessary repetition.

Finally, an embodiment example of a coupling membrane 8 which can likewise be used in the present device 1 and is already shown in FIG. 1 is shown in FIG. 7. This coupling membrane 8 in cross section as one can recognise particular clearly in FIG. 1 has the shape of a truncated cone. A centring point 40 which for the user simplifies the positioning of the device 1 onto the body of the patient is formed centrally on the truncated cone area. The lines or grooves 42 which are formed on the inclined sides 41 of the cone simplify the positioning and guiding of the device 1 onto the body of a patient for a user.

As is finally to be recognised in FIG. 1, the truncated-cone-shaped coupling membrane 8 is hollow on the inside and the sound wave source 4 is shaped such that it can be brought into the hollow region 45. By way of this, the sound wave source 4 can be moved over a further distance or alternatively the construction height of the device 1 can be reduced in size given an equal displacement height, compared to a filled coupling membrane or a flat coupling membrane.

The truncated cone shape of the coupling membrane 8 further has the advantage that the patient merely needs to come into contact with the coupling membrane 8 which is formed from soft plastic, and not with the housing 2 of the device 1 which is formed from a hard plastic. This ensures a more pleasant feeling for the patient. Furthermore, unevenness on the patient surface is compensated, by which means in particular given large penetration depths, losses are avoided on account of the larger coupling surface. Moreover, it is only the membrane which has to be disinfected after a treatment, since it is only this which comes into contact with the patient.

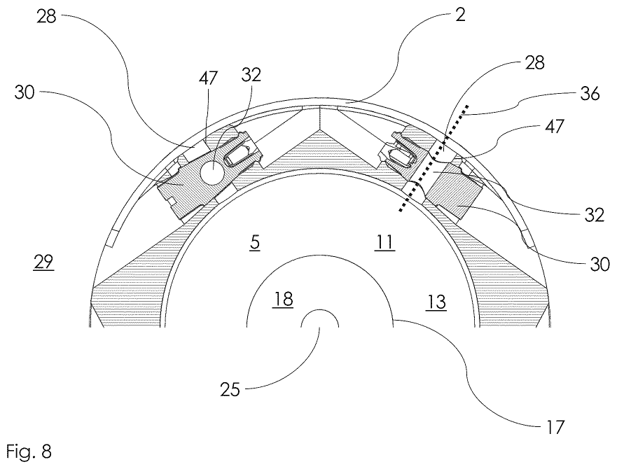

Finally, FIG. 8 shows a further embodiment example of a bleed opening 28 which can be used in the previously described embodiment example of a device 1. In the embodiment example which is shown in FIG. 8, two bleed openings 28 are provided, so that the fluid reservoir 5 can be filled for example via one of the bleed openings 28 and the air which is contained in the fluid reservoir 5 can escape via the other bleed opening 28 during the filling.

In the embodiment example, a closure means 30 in the form of a conical valve is arranged in each of the bleed openings 28, wherein the conical valve 30 is closed at the left side of FIG. 8 and the conical valve 30 is opened at the right side. In the embodiment example, the transition between the closed and opened conical valve is effected by way of a rotation of the conical valve 30 by a predefined angle of 90°. In the opened position, a bleed channel 32 in the cone valve 30 is aligned with the bleed opening 28, and the fluid reservoir 5 and in particular the bubble retention container 11 is in fluid communication with the surroundings 29. In the closed position which is defined by a stop, the bleed channel 32 is no longer aligned with the bleed opening 28.

A hose can be screwed into an inner thread 47 which is formed in the bleed opening, in order to fill the fluid reservoir 5. The conical valves 30 are sealing due to their truncated-cone-shaped outer shape and the tensile force which is mustered by a spring and which is in the direction of the truncated cone side which become narrower, so that in the closed state no gas or fluid can escape or flow in. In the opened state of the valve, the gas or the liquid flows to the outside or from the outside along the flow path only through the bleed channel 32 in the conical valve 30, said channel being envisaged for such.

While specific embodiments of the invention have been shown and described in detail to illustrate the application of the principles of the invention, it will be understood that the invention may be embodied otherwise without departing from such principles.

### LIST OF THE REFERENCE NUMERALS

1 device
2 housing
3 hand-piece, therapy source
4 sound wave source
5 fluid reservoir
6 coupling fluid
7 adjustment direction
8 coupling membrane
9 wall of the housing
10 coupling surface
11 bubble retention region
12 sound wave generation region
13 separating wall
14 wall of the fluid reservoir
15 first section of the separating wall
16 second section of the separating wall
17 free end of the separating wall
18 free space
19 intermediate space
20 collections of gas bubbles
21 extension direction of the coupling membrane and sound wave source
22 distance between the sound wave source and the coupling membrane
23 drive
24 spindle drive
25 axle
26 adjustment wheel
27 planetary gear
28 bleed opening
29 surroundings of the device
30 closure means
31 sealing means, O-ring
32 bleed channel
33 tip
34 special tool
35 channel
36 flow paths
37 recess, opening
38 recesses
39 engagement element
40 centring point
41 cone sides
42 grooves, guidance lines
43 spring
44 flow direction
45 hollow region
46 rotation axis of the adjustment wheel
47 thread

1. A device for the application of sound waves onto an animal and human body, the device comprising:
a housing in which a fluid reservoir for a coupling fluid is formed;
a sound wave source for generating sound waves;
a coupling membrane which in sections forms a wall of the fluid reservoir and is provided for coupling the sound waves which are generated by the sound wave source, into a human or animal body; and
a separating wall,
wherein the sound wave source is arranged in a sound wave generation region of the fluid reservoir which at least in sections is delimited by the coupling membrane,
wherein the fluid reservoir comprises a bubble retention region for holding back gas bubbles, said bubble retention region being in fluid communication with the sound wave generation region and being spatially separated from the sound wave generation region partly by way of the separating wall, and
wherein the sound wave source is arranged between the bubble retention region and the coupling membrane and is aligned such that sound waves which are generated by the sound wave source are delivered away from the bubble retention region and towards the coupling membrane.

2. A device according to claim 1, wherein the separating wall is formed by an undercut which projects out of the wall of the fluid reservoir.

3. A device according to claim 1 wherein the separating wall extends annularly away from the wall of the fluid reservoir.

4. A device according to claim 1, wherein the separating wall comprises a first section and a second section, wherein the first section extends between the sound wave source and the bubble retention region, wherein the second section connects onto the first section and wherein the second section extends away from the sound wave source and the coupling membrane.

5. A device according to claim 1, further comprising a closure means, wherein the wall of the fluid reservoir comprises a bleed opening through which the fluid reservoir can be bled, so that gas bubbles can escape out of the fluid reservoir through the bleed opening and wherein the closure means is configured to close the bleed opening.

6. A device according to claim 5, wherein the bleed opening runs out into the bubble retention region.

7. A device according to claim 5, wherein the bleed opening and the closure means are configured such that the closure means is brought into the bleed opening into a closure position for closing the bleed opening and is positioned only partly removed from the bleed opening in a bleed position for bleeding the fluid reservoir.

8. A device according to claim 5, wherein the closure means is configured such that the bleeding is effected through the closure means.

9. A device according to claim 5, further comprising a sealing means which is arranged in the bleed opening such that in a closure position, for closing the bleed opening, the closure means is in sealing contact with the sealing means and that in a bleed position, for bleeding the fluid reservoir, a bleeding through an intermediate space between the sealing means and the closure means is possible.

10. A device according to claim 5, wherein the fluid reservoir can be filled with coupling fluid through the bleed opening.

11. A device according to claim 5, wherein the fluid reservoir comprises an additional filling opening, via which the fluid reservoir can be filled with coupling fluid.

12. A device according to claim 1, wherein the sound wave source is movably arranged in the fluid reservoir such that a distance between the sound wave source and the coupling membrane can be changed, wherein an intermediate space between the sound wave source and the coupling membrane is filled with coupling fluid on operation of the device.

13. A device according to claim 12, further comprising a drive for changing the distance between the sound wave source and the coupling membrane.

14. A device according to claim 13, wherein the drive comprises an adjustment wheel which is configured such that the distance between the sound wave source and the coupling membrane can be changed by way of a manual rotation of the adjustment wheel.

15. A device according to claim 14, further comprising a spring-loaded engagement element which is biased in a direction of the adjustment wheel, and wherein the adjustment wheel comprises a plurality of indentations which are arranged at predefined distances,
wherein the engagement element and the indentations are arranged and configured such that depending on a position of the adjustment wheel, the engagement element engages into the indentations, so that an additional force is necessary in order to bring the engagement element out of engagement with the indentation by way of rotating the adjustment wheel,
wherein each position of the adjustment wheel in which the engagement element engages with one of the indentations corresponds to a predefined distance between the sound wave source and the coupling membrane.

16. A device according to claim 12, wherein the coupling membrane forms a bulge of the fluid reservoir away from the sound wave source, into which bulge the sound wave source can be moved.

17. A device according to claim 12, wherein the coupling membrane has a hollow truncated cone shape which tapers in a direction away from the sound wave source and projects out of the housing of the device.

18. A device according to claim 13,, wherein the drive comprises an actuator, which comprises a spindle drive.

19. A device according to claim 13, wherein the drive further comprises a planetary gear configured to cooperate with the adjustment wheel such that a rotation of the adjustment wheel is converted into a change of the distance between the sound wave source and the coupling membrane.

20. A device according to claim 15,wherein the engagement element is biased radially away from a rotation axis of the adjustment wheel towards the adjustment wheel or wherein the engagement element is biased parallel to the rotation axis of the adjustment wheel towards the adjustment wheel.
